(19) United States
(12) Patent Application Publication
(10) Pub. No.: US 2004/0147045 A1
(43) Pub. Date: Jul. 29, 2004

(54) Signal molecule arrays

(75) Inventor: Bryce P. Nelson, Madison, WI (US)

Correspondence Address: J. Mitchell Jones, MEDLEN & CARROLL, LLP, Suite 350, 101 Howard Street, San Francisco, CA, 94105, US

(73) Assignee: GenTel BioSurfaces, Inc., Madison, WI

(21) Appl. No.: 10/695,552

(22) Filed: Oct. 28, 2003

Related U.S. Application Data
(60) Provisional application No. 60/422,053, filed on Oct. 29, 2002.

(51) Int. Cl. G01N 33/543

(52) U.S. Cl. 436/518; 436/525

(57) ABSTRACT

The present invention relates to novel methods for the analysis of interactions of target molecules and cells. In particular, the present invention relates to compositions and methods for the detection of molecules involved in various aspects of cellular development. The present invention further provides methods of screening molecules for association with cellular development.

This application claims the benefit of U.S. provisional application 60/422,053, filed Oct. 29, 2002.

### FIELD OF THE INVENTION

The present invention relates to novel methods for the analysis of interactions between cells and molecules. In particular, the present invention relates to compositions and methods for the identification of molecules participating in cellular processes.

### BACKGROUND

A mature cell may be understood as a “steady state” system. A cell's DNA is constantly producing sets of mRNA, which produce cellular and extracellular proteins. As the proteins function, they are also degraded and replaced by new proteins. A mature cell is typically so well balanced that it neither grows, shrinks, nor changes in function. However, the process a cell undergoes upon its specialization is extremely dynamic and is of great interest to the fields of medicine, pharmacology, and empirical biology.

The dynamics of a cell is best understood by examining the course of its life. As a new cell is formed (e.g., either from the division of one cell into two, or from the fusion of sperm and egg cells), developmental processes begin. These developmental processes are different for each kind of cell and are “preprogrammed” in the cell's DNA. At various stages in a cell's life, different cellular or extracellular proteins are interacting with the cell and further facilitate cellular development.

Of particular value in the study of cellular development are stem cells. Stem cells have the ability to divide for indefinite periods in culture and to give rise to specialized cells. An understanding of how stem cells are induced to form a particular cell would assist in understanding organism development. In addition, understanding the mechanisms behind stem cell development would assist in the pursuit of cell based medical therapies.

Currently, numerous methods exist which are aiding in the study of cellular development. The analysis of proteins in histological sections and other cytological preparations is routinely performed using the techniques of histochemistry, immunohistochemistry, or immunofluorescence. By performing immunofluorescence with antibodies labeled with different colors, it has been possible to detect simultaneously 2, 3, or even 4 different antigens present in cellular material. In the future, time-resolved fluorescence may permit the extension of immunofluorescence methods to the detection of 6 to 12 different antibodies simultaneously. In the pursuit of identifying wide scale cellular developmental mechanisms, however, this method is too laborious and time consuming.

The study of cells by measuring the identity and concentration of a relatively large number of proteins simultaneously (referred to as proteomics) is currently a very time-consuming task. Two-dimensional (2D) gel electrophoresis is the most powerful tool for studying the expression of multiple proteins, but this technique is not readily adaptable to in situ cell analysis. Typically, many thousands of cells are required to perform a single 2D gel analysis. In order to identify different protein expression profiles in heterogeneous tissue samples, one would need the capability to analyze the proteins expressed in a small number of cells.

Mass spectroscopy is another powerful technique for protein analysis. However, the direct analysis of proteins present in samples containing small numbers of cells is not possible with prior mass spectroscopy technology, due to insufficient sensitivity. A minimum of 10,000 cells is required for mass spectroscopic analysis of tissue samples using prior technology.

What is needed are devices and methods for rapidly detecting simultaneous interactions between thousands of proteins and a particular cell. In particular, a method that can simultaneously screen multiple molecules for their potential to act as differentiation and developmental signals is needed.

### SUMMARY

The present invention relates to novel methods for the analysis of interactions between cells and molecules. In particular, the present invention relates to compositions and methods for the identification of molecules participating in cellular processes.

In some embodiments, the present invention comprises an arrayed solid surface. The solid surface is comprised of a plurality of addressable target molecules, with the solid surface in contact with a cellular product. In certain embodiments, the target molecules are selected from a group consisting of natural or synthetic oligonucleotides, viruses, polypeptides, antibodies, naturally occurring drugs, synthetic drugs, pollutants, allergens, affector molecules, growth factors, chemokines, nucleic acids, cytokines, and lymphokines. In other embodiments, the plurality of target molecules are selected from a group consisting of cell initiation molecules, cell differentiation molecules, cell morphogenesis molecules, and cell maintenance molecules. In some embodiments, the plurality of target molecules are suspected of interacting with whole cell solutions, lysed cell solutions, subcellular compartment solutions, or mixes therewith. In other embodiments, the plurality of target molecules are known to interact with whole cell solutions, lysed cell solutions, subcellular compartment solutions, or mixes therewith.

In arrayed solid surface embodiments, the cellular product may comprise whole cell solutions, lysed cell solutions, subcellular compartment solutions, or a cellular mixture comprising combinations of whole cell solutions, lysed cell solutions, or subcellular compartment solutions.

In other arrayed solid surface embodiments, the cellular product may comprise whole stem cell solutions. Such whole stem cell solutions are selected from the group consisting of whole human stem cell solutions, whole murine stem cell solutions, whole porcine stem cell solutions, and whole primate stem cell solutions. In other embodiments, the cellular product may comprise lysed stem cell solutions. Such lysed cell solutions are selected from the group consisting of lysed human stem cell solutions, lysed murine stem cell solutions, lysed porcine stem cell solutions, and lysed primate stem cell solutions. In yet other embodiments, the cellular product may comprise stem cell subcellular compartment solutions. Such stem cell subcellular compartment solutions are selected from the group consisting of human stem cell subcellular compartment solutions, murine stem cell subcellular compartment solutions, porcine stem cell subcellular compartment solutions, and primate stem cell subcellular compartment solutions. In further embodiments, the cellular product may comprise a stem cell cellular mixture. Such stem cell cellular mixtures are comprised of combinations of whole stem cell solutions, lysed stem cell solutions, or stem cell subcellular compartment solutions. In yet other embodiments, the stem cell cellular mixture is selected from human stem cells, murine stem cells, porcine stem cells and primate stem cells.

In some arrayed solid surface embodiments, the solid surface is configured for label free detection. In other embodiments, the solid surface may be an SPR surface, or an SPR prism. In further embodiments, the solid surface comprises a plurality of one dimensional or two dimensional microfluidics channels. Such microfluidics channels may be one dimensional, two dimensional. In other embodiments, the solid surface may comprise a plurality of etched microchannels.

In other solid surface array embodiments, the plurality of target molecules comprises at least 2 unique target molecules. In further embodiments, the plurality of target molecules comprises at least 50 unique target molecules. In other embodiments, the plurality of target molecules comprises between 2 and 50 target molecules. In yet other embodiments, the plurality of target molecules comprises at least 1000 unique target molecules. In further embodiments, the plurality of target molecules comprises between 10 and 1000 unique target molecules. In further embodiments, the plurality of target molecules comprises at least 10000 unique target molecules. In still further embodiments, the plurality of target molecules comprises between 10 and 10000 unique target molecules.

In other embodiments, the present invention is a method to identify molecules that interact with a cell component. In such a method, an arrayed surface comprising an array of addressable target molecules is contacted with a cellular product. In other embodiments, the method may also comprise the step of detecting the presence or absence of an interaction between the cellular product and the arrayed target molecules. The target molecules are selected from the group consisting of natural or synthetic oligonucleotides, viruses, polypeptides, antibodies, naturally occurring drugs, synthetic drugs, pollutants, allergens, affector molecules, growth factors, chemokines, nucleic acids, cytokines, and lymphokines. In other embodiments, the target molecules comprise proteins, peptides, and small molecules.

In some embodiments used to identify molecules that interact with cell components, the cellular product may comprise whole cell solutions, lysed cell solutions, subcellular compartment solutions, or a cellular mixture comprising combinations of whole cell solutions, lysed cell solutions, or subcellular compartment solutions.

In embodiments used to identify molecules that interact with cell components, the cellular product may comprise whole cell solutions, lysed cell solutions, subcellular compartment solutions, or a cellular mixture comprising combinations of whole cell solutions, lysed cell solutions, or subcellular compartment solutions.

In other embodiments used to identify molecules that interact with a cell component, the cellular product may comprise whole stem cell solutions. Such whole stem cell solutions are selected from the group consisting of whole human stem cell solutions, whole murine stem cell solutions, whole porcine stem cell solutions, and whole primate stem cell solutions. In other embodiments, the cellular product may comprise lysed stem cell solutions. Such lysed cell solutions are selected from the group consisting of lysed human stem cell solutions, lysed murine stem cell solutions, lysed porcine stem cell solutions, and lysed primate stem cell solutions. In yet other embodiments, the cellular product may comprise stem cell subcellular compartment solutions. Such stem cell subcellular compartment solutions are selected from the group consisting of human stem cell subcellular compartment solutions, murine stem cell subcellular compartment solutions, porcine stem cell subcellular compartment solutions, and primate stem cell subcellular compartment solutions. In further embodiments, the cellular product may comprise a stem cell cellular mixture. Such stem cell cellular mixtures are comprised of combinations of whole stem cell solutions, lysed stem cell solutions, or stem cell subcellular compartment solutions. In yet other embodiments, the stem cell cellular mixture is selected from human stem cells, murine stem cells, porcine stem cells and primate stem cells.

In embodiments used to identify molecules that interact with a cell component, the solid surface is configured for label free detection. In other embodiments, the solid surface may be an SPR surface, or an SPR prism. In further embodiments, the solid surface comprises a plurality of one dimensional or two dimensional microfluidics channels. Such microfluidics channels may be one dimensional, two dimensional. In other embodiments, the solid surface may comprise a plurality of etched microchannels.

In other embodiments used to identify molecules that interact with a cell component, the plurality of target molecules comprises at least 2 unique target molecules. In further embodiments, the plurality of target molecules comprises at least 50 unique target molecules. In other embodiments, the plurality of target molecules comprises between 2 and 50 target molecules. In yet other embodiments, the plurality of target molecules comprises at least 1000 unique target molecules. In further embodiments, the plurality of target molecules comprises between 10 and 1000 unique target molecules. In further embodiments, the plurality of target molecules comprises at least 10000 unique target molecules. In still further embodiments, the plurality of target molecules comprises between 10 and 10000 unique target molecules.

The present invention is also contemplated to act as a system comprising an arrayed solid surface in which the solid surface has a plurality of addressable target molecules in contact with a cellular product. Other system embodiments may also comprise a detection apparatus configured to detect the presence or absence of an interaction between the cellular product and the target molecules. In further embodiments, the detection apparatus is in communication with said arrayed solid surface. In other furthered embodiments, the detection apparatus is configured for detecting interactions between said arrayed solid surface and said cellular product. Additional embodiments may also comprise an instruction manual.

In some system embodiments, the target molecules are selected from the group consisting of natural or synthetic oligonucleotides, viruses, polypeptides, antibodies, naturally occurring drugs, synthetic drugs, pollutants, allergens, affector molecules, growth factors, chemokines, nucleic acids, cytokines, and lymphokines. In other embodiments, the target molecules comprise proteins, peptides, and small molecules.

In some system embodiments, the cellular product may comprise whole cell solutions, lysed cell solutions, subcellular compartment solutions, or a cellular mixture comprising combinations of whole cell solutions, lysed cell solutions, or subcellular compartment solutions.

In system embodiments, the cellular product may comprise whole cell solutions, lysed cell solutions, subcellular compartment solutions, or a cellular mixture comprising combinations of whole cell solutions, lysed cell solutions, or subcellular compartment solutions.

In other system embodiments, the cellular product may comprise whole stem cell solutions. Such whole stem cell solutions are selected from the group consisting of whole human stem cell solutions, whole murine stem cell solutions, whole porcine stem cell solutions, and whole primate stem cell solutions. In other embodiments, the cellular product may comprise lysed stem cell solutions. Such lysed cell solutions are selected from the group consisting of lysed human stem cell solutions, lysed murine stem cell solutions, lysed porcine stem cell solutions, and lysed primate stem cell solutions. In yet other embodiments, the cellular product may comprise stem cell subcellular compartment solutions. Such stem cell subcellular compartment solutions are selected from the group consisting of human stem cell subcellular compartment solutions, murine stem cell subcellular compartment solutions, porcine stem cell subcellular compartment solutions, and primate stem cell subcellular compartment solutions. In further embodiments, the cellular product may comprise a stem cell cellular mixture. Such stem cell cellular mixtures are comprised of combinations of whole stem cell solutions, lysed stem cell solutions, or stem cell subcellular compartment solutions. In yet other embodiments, the stem cell cellular mixture is selected from human stem cells, murine stem cells, porcine stem cells and primate stem cells.

In other system embodiments, the solid surface is configured for label free detection. In other embodiments, the solid surface may be an SPR surface, or an SPR prism. In further embodiments, the solid surface comprises a plurality of one dimensional or two dimensional microfluidics channels. Such microfluidics channels may be one dimensional, two dimensional. In other embodiments, the solid surface may comprise a plurality of etched microchannels.

In other system embodiments, the plurality of target molecules comprises at least 2 unique target molecules. In further embodiments, the plurality of target molecules comprises at least 50 unique target molecules. In other embodiments, the plurality of target molecules comprises between 2 and 50 target molecules. In yet other embodiments, the plurality of target molecules comprises at least 1000 unique target molecules. In further embodiments, the plurality of target molecules comprises between 10 and 1000 unique target molecules. In further embodiments, the plurality of target molecules comprises at least 10000 unique target molecules. In still further embodiments, the plurality of target molecules comprises between 10 and 10000 unique target molecules.

The present invention is also contemplated to serve as a process toward identifying novel compounds. Such a process utilizes a solid surface with an array of addressable putative signal molecules in contact with a cellular product. In other embodiments, the process also contains the step of detecting the presence or absence of an interaction of the cellular product with at least one of the putative signal molecules using a detection apparatus. In further embodiments, a additional step is used to determine the composition of the at least one target molecule interacting with the cellular product by reference to the address of the target molecule on the addressable array.

In other process embodiments, the cellular product is a solution of whole cells, lysed cell solutions, subcellular compartment solution, or mixes therewith. In some embodiments, the process further comprises the step of culturing the whole cell following said contact with said solid surface.

In some embodiments, the process is furthered by measuring changes in the internal state of the cell product. In more detailed embodiments, the measured change in internal state is a change selected from the group consisting of a change in phosphorylation, release of calcium, sequestration of calcium, proteolysis, protein synthesis, cyclic nucleotide production, inositol triphosphate production, gene transcription, and mRNA degradation. Alternatively, in other embodiments, the measured change in internal state is selected from the group consisting of activation of the cell division cycle, repression of the cell division cycle, activation of a cell death pathway, and repression of a cell death pathway.

Definitions

As used herein, the term “solid surface” or “solid support” refers to any solid surface suitable for the attachment of biological molecules and the performance of molecular interaction assays. Surfaces may be made of any suitable material (e.g., including, but not limited to, metal, glass, and plastic) and may be modified with coatings (e.g., metals or polymers).

As used herein, the term “SPR surface” refers to a solid surface that is suitable for use in SPR detection. In some embodiments, “SPR surfaces” are “SPR prisms.”

As used herein, the term “target molecule” refers to a molecule capable of associating or suspected of associating with a “cellular product.” Examples of target molecules include, but are not limited to, oligonucleotides (e.g., containing a particular DNA binding domain recognition sequence), viruses, polypeptides, antibodies, naturally occurring drugs, synthetic drugs, pollutants, allergens, affector molecules, growth factors, synthetic molecules, chemokines, cytokines, and lymphokines. In some embodiments, target molecules are attached to a solid surface (e.g., array).

As used herein, the term “small molecules” refer to items within the set of target molecules. Examples of small molecules include, but are not limited to, naturally occurring drugs, synthetic drugs, pollutants, allergens, affector molecules, growth factors, chemokines, cytokines, and lymphokines.

In contrast, “large molecules” or “biological macromolecule” refers to large molecules (e.g., polymers) typically found in living organisms. Examples include, but are not limited to, proteins, nucleic acids, viruses, lipids, carbohydrates, oligonucleotides, and antibodies.

As used herein, the term “cellular product” refers to intact cells, lysed cells, or tissues. Examples of cellular products include, but are not limited to, whole cell solutions, lysed cell solutions, subcellular fragment solutions, whole tissues, partial tissues, tissue solutions, cellular solutions, synthetic cells, synthetic tissues, or mixes therewith.

As used herein, the term “affector” refers to any molecule that causes a change of state in a cell. Examples include, but are not limited to, molecules inducing initiation of cell differentiation, molecules inducing cell death, molecules inducing cell division, molecules repressing cell division, molecules inducing changes in transcription rates of genes or sets of genes, or molecules inducing accumulation of proteins or degradation thereof.

As used herein, the term “cell initiation” refers to a cell at or near a state of formation. Examples include, but are not limited to, the state of a cell upon its division from a parent cell, and the state of an unfertilized ova upon fertilization.

As used herein, the term “cell initiation molecules” refers to molecules associated with initiation of cell development. Examples include, but are not limited to, proteins and hormones.

As used herein, the term “cell differentiation” refers to a cell at or near a state of commitment to a particular development fate. Examples include, but are not limited to, the state of an embryonic cell upon commitment to a various specific cell type (e.g., keratinocyte, erythrocyte, lens cell, B lymphocyte, T lymphocyte, melanocyte, pancreatic islet cells, Leydig cells, chondrocytes, osteoblasts, myocytes, hepatocytes, neurons).

As used herein, the term “cell differentiation molecules” refers to molecules associated with differentiation of cell development. Examples include, but are not limited to, proteins and hormones.

As used herein, the term “cell morphogenesis” refers to a cell at or near a state of associating with other cells to develop into a complex grouping. Examples include, but are not limited to, the state of cell upon associating with other cells to form tissue, and the state of a cell upon associating with other cells to form an organ.

As used herein, the term “cell morphogenesis molecules” refers to molecules associated with morphogenesis of cell development. Examples include, but are not limited to, proteins and hormones.

As used herein, the term “cell maintenance” refers to a cell at or near a state of maturation. Examples include, but are not limited to, the state of a cell upon maturation into an adult cell, and the state of a cell upon initiation of cell death.

As used herein, the term “cell maintenance molecules” refers to molecules associated with maintenance of cell development. Examples include, but are not limited to, proteins and hormones.

As used herein, the term “whole cell solution” refers to intact cells. Examples include, but are not limited to intact vascular endothelial cells, intact stem cells (e.g., epidermal basal cells, osteoprogenitor cells, synthetic stem cells), intact hepatocytes, intact red blood cells, and intact synthetic cells.

As used herein, the term “lysed cell solution” refers to a fractioned cell. Examples include, but are not limited to, a portion of a hepatocyte, a homogenized stem cell (e.g., epidermal basal cells, osteoprogenitor cells, synthetic stem cells), a homogenized hepatocyte, a hepatocyte solution, and a suspended synthetic cell.

As used herein, the term “subcellular fragment solution” refers to a portion of a cell. Examples include, but are not limited to, stem cell organelles (e.g., a complete osteoprogenitor cell nucleus, a part of an osteoprogenitor cell nucleus, an osteoprogenitor cell extracellular matrix, an osteoprogenitor cell nuclear membrane, an osteoprogenitor cell golgi apparatus, a homogenized osteoprogenitor cell endoplasmic reticulum/golgi apparatus mixture, and synthetic stem cell subcellular compartments), a hepatocyte nucleus, a suspended hepatocyte nuclear membrane, a homogenized hepatocyte nucleus, a hepaocyte nuclear membrane solution, a part of an epithelial cell nucleus, a homogenized contractile cell golgi apparatus, a suspended thyroid gland secreting cell golgi apparatus, a exocrine gland striated cell golgi apparatus solution, a lung lining cell endoplasmic reticulum/hepatoctye golgi apparatus mixture, and synthetic subcellular fragment solutions.

As used herein, the term “cell adhesion assay” refers to any method of analyzing associations (e.g., binding) between a “cellular product” and “target molecules.” Examples include, but are not limited to, fluorescence based detection, SPR detection, and array based detection.

As used herein, the term “SPR capable metal film” refers to any metallic film that is suitable for use in SPR detection. Examples include, but are not limited to, gold, silver, chrome, and aluminum.

As used herein, the term “substrate” refers to any material with a surface that may be coated with a film.

As used herein, the phrase “coated with a film” in regard to a substrate refers to a situation where at least a portion of a substrate surface has a film attached to it (e.g., through covalent or non-covalent attachment).

As used herein, the term “microarray” refers to a solid surface comprising a plurality of addressed biological macromolecules (e.g., nucleic acids or antibodies). The location of each of the macromolecules in the microarray is known, so as to allow for identification of the samples following analysis.

As used herein, the term “array of target molecules” or “array of target molecule binding targets” refers to a microarray of target molecules that are known to, or are suspected of, associating with a “cellular product.”

As used herein, the term “disposable arrayed SPR prism” refers to a prism that is suitable for use in SPR detection, comprises an arrayed surface (e.g., a microarray), and is not intended to be reused for multiple detection assays. In some embodiments, the disposable arrayed prisms are those disclosed herein.

As used herein, the term “coated on one face” when used in reference to an SPR prism, refers to a prism with a coating on one of the main faces of the prism. For example, in some embodiments, triangular prisms are coated on the upward facing surface. The term “face” is not intended to encompass the small facets on each face of a prism that reflect light.

As used herein, the term “microfluidics channels” or “etched microchannels” refers to three-dimensional channels created in material deposited on a solid surface. In some embodiments, microchannels are composed of a polymer (e.g., polydimethylsiloxane). Exemplary methods for constructing microchannels include, but are not limited to, those disclosed herein.

As used herein, the term “one-dimensional line array” refers to parallel microfluidic channels on top of a surface that are oriented in only one dimension.

As used herein, the term “two dimensional arrays” refers to microfluidics channels on top of a surface that are oriented in two dimensions. In some embodiments, channels are oriented in two dimensions that are perpendicular to each other.

As used herein, the term “microchannels” refers to channels etched into a surface. Microchannels may be one-dimensional or two-dimensional.

The term “sample” as used herein is used in its broadest sense and includes, but is not limited to, environmental, industrial, and biological samples. Environmental samples include material from the environment such as soil and water. Industrial samples include products or waste generated during a manufacturing process. Biological samples may be animal, including, human, fluid (e.g., blood, plasma and serum), solid (e.g., stool), tissue, liquid foods (e.g., milk), and cell lysates (e.g., cultured cell lysates).

The term “test compound” refers to any chemical entity, pharmaceutical, drug, and the like that is suspected of altering the affinity of a cell product for its target sequence. Test compounds comprise both compounds known to alter such interactions, and those suspected to. A test compound can be determined to be active in altering binding interactions by screening using the screening methods of the present invention.

The term “signal” as used herein refers to any detectable effect, such as would be caused or provided by an assay reaction. For example, in some embodiments of the present invention, signals are SPR or fluorescent signals.

As used herein, the term “label free detection” refers to the detection of a binding interaction between unlabeled cell products and binding targets. Methods of label free detection include, but are not limited to, those disclosed herein.

As used herein, the term “detection apparatus” refers to an apparatus configured for the detection of an interaction between a cell product and a nucleic acid target. In some embodiments, detection apparatus are configured for “label free detection.” In other embodiments, they are configured for detection of a label (e.g., on or within a cell product).

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

As used herein, the term “stem cells” refers to cells competent to undergo more than one developmental fate.

As used herein, the term “cell culture” refers to any in vitro maintenance of living cells. Included within this term are continuous cell lines (e.g., with an immortal phenotype), primary cell cultures, finite cell lines (e.g., non-transformed cells), and any other cell population maintained in vitro, including oocytes and embryos. The cells maintained in vitro may grow and/or divide or differentiate into a new cell type but in any case remain metabolically active.

As used herein, the term “genome” refers to the genetic material (e.g., chromosomes) of an organism.

The term “nucleotide sequence of interest” refers to any nucleotide sequence (e.g., RNA or DNA), the manipulation of which may be deemed desirable for any reason (e.g., treat disease, confer improved qualities, expression of a protein of interest in a host cell, expression of a ribozyme, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The terms “homology” and “percent identity” when used in relation to nucleic acids refer to a degree of complementarity. There may be partial homology (i.e., partial identity) or complete homology (i.e., complete identity). A partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe (i.e., an oligonucleotide which is capable of hybridizing to another oligonucleotide of interest) will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) to the single-stranded nucleic acid sequence under conditions of low stringency as described above.

As used herein the term, the term “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “purified” refers to molecules, either nucleic or amino acid sequences that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

In particular, the term “gene” refers to the full-length nucleotide sequence. However, it is also intended that the term encompass fragments of the sequence, as well as other domains within the full-length nucleotide sequence. Furthermore, the terms “nucleotide sequence” or “polynucleotide sequence” encompasses DNA, cDNA, and RNA (e.g., mRNA) sequences.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

### DETAILED DESCRIPTION

The present invention provides compositions and methods for the detection of interactions between whole cell solutions, lysed cell solutions, subcellular compartments, or mixes therewith, and target molecules. The present invention further provides methods of screening molecules for an ability to initiate cell differentiation, morphogenesis, or maintenance. The array-based methods of the present invention are able to overcome many of the disadvantages of previous methods. For example, in some embodiments an array-based method using the present invention provides SPR imaging that drastically simplifies the procedure for screening various polypeptides or other target molecules for a suspected capability. The methods of the present invention further provide improved methods of identifying molecules that initiate stem cell differentiation, morphogenesis, and maintenance.

I. Solid Supports

In some preferred embodiments, the present invention utilizes solid supports for performing cell adhesion assays. The present invention is not limited to a particular solid support. Any number of solid supports may be utilized, including, but not limited to, protein or DNA “chips” composed of any number of suitable materials, and SPR (e.g., metal) surfaces. In some preferred embodiments, solid supports contain arrays of biological macromolecules (e.g., nucleic acids or proteins).

A. Chips

In some embodiments, the solid support is a “chip.” Chips may be made of any suitable material including, but not limited to, metal, plastic, polymer, and glass. Several commercial sources for chips, with and without already arrayed biological molecules, exist (See e.g., the below discussion of arrays). Commercial sources include, but are not limited to, Motorola, Schaumburg, Ill.; ACLARA BioSciences, Inc., Hayward, Calif.; Agilent Technologies Inc., Palo Alto, Calif.; Aviva Biosciences Corp., Dan Diego, Calif.; Caliper Technologies Corp., Palo Alto, Calif.; Clontech, Palo Alto, Calif.; Corning, Acton, Mass.; Gene Logic Inc., Columbia, Md.; Hyseq Inc., Sunnyvale, Calif.; Incyte Genomics, Palo Alto, Calif.; Micronics Inc., Redmond, Wash.; Mosaic Technologies, Waltham, Mass.; OriGene Technologies, Rockville, Md.; Packard Instrument Corp., Meriden, Conn.; Rosetta Inpharmatics, Kirkland, Wash.; and Sequenom, San Diego, Calif.

B. SPR Surfaces

In other embodiments, the solid support is an SPR surface. Surface Plasmon Resonance techniques involve a surface coated with a thin film of a conductive metal, such as gold, silver, chrome or aluminum, in which electromagnetic waves, called Surface Plasmons, can be induced by a beam of light incident on the metal glass interface at a specific angle called the Surface Plasmon Resonance angle. Modulation of the refractive index of the interfacial region between the solution and the metal surface following binding of the captured macromolecules causes a change in the SPR angle which can either be measured directly or which causes the amount of light reflected from the underside of the metal surface to change. Such changes can be directly related to the mass and other optical properties of the molecules binding to the SPR device surface. Several biosensor systems based on such principles have been disclosed (See e.g., WO 90/05305).

Generally, in a Kretschman-configuration SPR device, a glass cover slip or slide of appropriate refractive index is coated with a thin (on the order of 50 nm) SPR-capable metal layer. This metal surface is then chemically patterned, and probe molecules are attached to the pattern features. The patterning can be either a basic grid-like array, or microfluidic channels can be overlaid onto the surface for probe deposition and sample application. This gold coated, patterned slide is then optically linked to a prism. This linkage is accomplished by placing a thin film of index-matching fluid between the prism and the slide. A sample solution is then passed over the probes arrayed on the surface. Interaction of an analyte in the solution with a probe molecule on the surface is detected as a change in refractive index. Importantly, SPR detection is label-free.

In some embodiments, a disposable SPR prism is utilized. The prism may be made of any suitable material including, but not limited to, glass and silica. In preferred embodiments, prisms are made of a high refractive index material. Preferred materials are those whose SPR minimum falls within an angle range. The range can be determined by applying known formulas (See e.g., Hansen, W. N. Journal of the Optical Society of America 53(3):380-390). For example, in some embodiments, prisms are made from a material including, but not limited to, BK-7 glass, SFL-6 glass, and preferably SF-10 glass.

In some embodiments, the prisms are coated on one face with an SPR-capable metal layer. The present invention is not limited to a particular type of metal. Any metal that is suitable for use in SPR may be utilized including, but not limited to, gold, silver, chrome or aluminum. The thickness of the metal film is not overly critical insofar as the film is uniformly applied and will function in SPR imaging analysis. In preferred embodiments, a film of about 450 A thick is used. In preferred embodiments, gold is utilized as the SPR capable film to coat the prisms.

In some embodiments, the metal (e.g., gold) layer is chemically patterned for attachment of molecular probes (e.g., biomolecules). The present invention is not limited to a particular biological macromolecule. A variety of biological macromolecules are contemplated including, but not limited to, DNA, proteins, carbohydrates, lipids and amino acids.

C. Arrays

In some embodiments, solid surfaces are chemically patterned for attachment of biological macromolecules (e.g., nucleic acids or proteins). In some embodiments, the present invention further provides solid supports comprising arrays of biological macromolecules. In preferred embodiments, arrays comprise at least 50, preferably at least 100, even more preferably at least 1000, still more preferably, at least 10,000, and yet more preferably, at least 100,000 distinct biological macromolecules. In preferred embodiments, each distinct biological macromolecule is addressed to a specific location on the array. This allows simultaneous screening of all the arrayed molecules, and allows for the immediate identification of any molecule that interacts with a cell product. In preferred embodiments, each addressable location is larger than 25, and preferably, larger than 50 microns.

The present invention is not limited to a particular method of fabricating or type of array. Any number of suitable chemistries known to one skilled in the art may be utilized. In preferred embodiments, the target molecules are attached to the substrate by a cleavable disulfide bond.

1. Amine Modified Surface Arrays

In some preferred embodiments, the method of generating arrays described in U.S. Pat. No. 6,127,129 (herein incorporated by reference) is utilized. In the first step of the method, a monolayer of a thiol is self-assembled from an ethanolic solution onto a solid support. In some embodiments, the support is a glass slide. In more preferred embodiments, the support is a solid support which has been coated with a thin noble-metal film. The present invention is not limited to a particular thiol. A variety of lengths and positions of attachment of the thiol group are contemplated as being suitable for use in the present invention. In some preferred embodiments, long chain (e.g., 11 carbon) alkanethiols are utilized. In other embodiments, branched or cyclic thiols are utilized.

In some embodiments, amine (e.g., MUAM) or carboxylic acid terminated (e.g., MUA), hydroxyl terminated (e.g., MUD), or MUAM modified to be thiol terminated are utilized. In some particularly preferred embodiments, an co-modified alkanethiol, preferably an amine-terminated alkanethiol, most preferably 11-mercaptoundecylamine (MUAM), is utilized (See e.g., Thomas et al., J. Am. Chem. Soc. 117:3830 [1995]).

Self-assembled monolayers of co-modified alkanethiols on gold form well ordered, monomolecular films. However, if left exposed for extended periods of time, the terminal amine groups of amino-modified alkanthiols may react with CO₂ to form carbamate salts on the surface. Consequently, it is preferred that exposure of amino-terminated alkanethiol-coated substrates to CO₂ be minimized.

Next, the alkanethiol-covered surface is reacted with a reversible protecting group to create a hydrophobic surface. In certain embodiments utilizing an amine-modified alkanethiol such as MUAM, the protecting group is an amino protecting group, preferably 9-fluorenylmethoxycarbonyl (Fmoc). The present invention is not limited to an Fmoc protecting group. Any reversible protecting group may be utilized. Preferred protecting groups offer efficient protection, favorable (e.g., to biological molecules) deprotecting conditions, efficient deprotection, and are suitable for reactions on a surface. For example, in some embodiments, Thoc is utilized for the protection of alkanethiols.

Fmoc is a bulky, hydrophobic, base labile, amine protecting group routinely used in the solid phase synthesis of peptides. The choice of protecting group used is dependent in large measure upon the nature of the ω-modification made to the alkanethiol. If the ω-modification is the addition of a carboxyl group, a hydrophobic carboxy protecting group is preferred. Likewise, if the ω-modification is the addition of a hydroxyl or thiol group, a hydrophobic hydroxy or thiol protecting group, respectively, is preferred used. Any type of hydrophobic protecting group suitable for protecting the co-modification used on the alkanethiol can be utilized in the present invention. Numerous such protecting groups, for any number of reactive moieties, such as amine, hydroxy, ester, carbamate, amides, ethers, thoioethers, thioesters, acetals, ketals and carboxy functionalities, are known to the art (See e.g., Frutos et al., Langmuir 16:2192 [2000]). For example, chloride derivatives of both Fmoc and trityl can be used to reversibly modify hydroxyl-terminated alkanethiols.

In some embodiments utilizing Fmoc protecting groups, the N-hydroxysuccinimide ester of Fmoc (Fmoc-NHS) is reacted with the terminal amine moiety of the MUAM molecule to form a stable carbamate (urethane) linkage, covalently attaching the Fmoc group to the surface.

Subsequently, the bond anchoring the alkanethiol to the metal substrate is selectively cleaved to yield a patterned surface of exposed metal. In some preferred embodiments, UV photopatterning is utilized to create the patterned surface. However, any suitable method of generating a patterned surface may be utilized. For example, in some embodiments, microcontact printing methods can also be used to yield a patterned surface. Using UV patterning, the surface is exposed through a quartz mask to UV radiation, which photo-oxidizes the gold-sulfur bond that anchors the alkanethiol monolayers to the surface. The surface is then rinsed, removing the photo-oxidized alkanethiol and leaving an array of bare metal pads surrounded by a hydrophobic MUAM+Fmoc background. Using photopatterning, features with dimensions as small as 50 nm have been achieved; using microcontact printing methods, arrays with features as small as about 100 nm are achievable.

The surface is next exposed to an alkanethiol solution (in some preferred embodiments, an ethanolic solution of MUAM) whereby the alkanethiol assembles into the bare gold regions producing a surface composed of hydrophilic alkanethiol pads surrounded by the hydrophobic blocked background. This difference in hydrophobicity between the reactive alkanethiol regions and the background is useful for the pinning of small volumes of aqueous biomolecule or cell solutions onto individual array locations.

Biological macromolecules are then covalently attached to the surface. The alkanethiol active pads are first exposed to a solution of a bifunctional linker. Preferred linkers are those capable of binding at one end to the alkanethiol surface and at the other end to the biological macromolecule to be immobilized to form the desired array. Any bifunctional (e.g., hetero or homo bifunctional) linker having these characteristics can be used in the present invention (See e.g., Smith et al., Langmuir 17:2502 [2001] and the Catalog of Pierce Chemical Company, Rockford, Ill.). Exemplary linkers include, but are not limited to, SSMCC, DSS, and PDITC.

The preferred bifunctional linker is sulfosuccinimidyl 4-(N-maleimidomethyl)cyclohexane-1-carboxylate (SSMCC), a heterobifunctional linker which contains both an N-hydroxysulfosuccinimide (NHSS) ester and a maleimide functionality. The NHSS ester end of the molecule reacts with the free amine groups on an amino-modified surface, such as the MUAM spots, creating pads terminated in maleimide groups, which are reactive towards thiols. Small volumes (0.08 to 0.1 L) of 1 mM solutions of 5′-thiol-modified biological macromolecules (e.g., DNA sequences) are then spotted at discrete array locations and react to form a covalent attachment to the surface. Using this technique, any number of biological macromolecules can be spotted at different array locations.

The protecting group (e.g., Fmoc) is next removed from the array surface. Preferably, this is accomplished by exposure to a 1M solution of the secondary amine, TAEA, in DMF. Many basic secondary amines can be used to remove Fmoc from the surface (e.g., including, but not limited to, 1M solutions of ethanolamine and piperidine). After the deprotection step, the array background has been converted back to the original alkanethiol surface.

In the final step of the array fabrication, the alkanethiol background is reacted with a compound to create a background that is resistant to the non-specific binding of proteins. The preferred compound for this purpose is PEG-NHS, although any compound that will selectively bind to the alkanethiol surface and inhibit non-selective protein binding can be used. In order to effectively monitor the binding of proteins to arrays of surface-bound biomolecules or cells, it is preferred that the array background prohibit the non-specific adsorption of protein molecules. Additional blocking groups include, but are not limited to, mixtures of PEG-terminated and other molecules (e.g., hydroxyl-terminated), different molecular weights of PEG molecules, polylysine, casein, BSA, and octadecane thiol (See e.g., Chapman et al., J. Am. Chem. Soc., 122:8303 [2000]).

2. Additional Arrays

In some embodiments, the solid support is first treated in order to create chemically reactive groups on the surface of the support. These chemically reactive groups serve as sites to which the target molecules can be attached. In certain embodiments, glass slides are treated with an aldehyde-containing silane reagent. In the case of arraying smaller proteins, the solid support is activated by first attaching BSA to the surface and then activating the BSA to obtain functionalized sites on the BSA protein. The smaller proteins are then attached to the functionalized sites on the BSA protein.

In some preferred embodiments, the chemically-active groups on the surface are thiol groups. The thiol groups can be used to attach thiol-containing target molecules through a covalent disulfide linkage (see, e.g., Smith et. al., Langmuir, 17:2502 (2001)). This linkage may later be cleaved, for example with dithiothreitol (DTT), thereby simultaneously releasing the target molecule from the surface and regenerating an active attachment site. The target molecule and adhering cell product may then be subjected to further analysis off of the array. The target molecule and cellular product may then be further dissociated and assayed after the interaction with the cellular product has occurred.

The process of making thiol-terminated surfaces is similar to the process described above for the creation of maleimide-terminated surfaces. The first step of the process is the creation of an amine-terminated self-assembled monolayer on an appropriate substrate, as above. In a preferred embodiment, the substrate is a gold coated slide, and the monolayer is comprised of 11-mercaptoundecylamine (MUAM). The monolayer is deposited onto the substrate from a 1.0 mM ethanolic solution for at least 24 h.

In preferred embodiments, creation of the thiol-activated surface is performed by reaction of the amino group of the MUAM monolayer with crosslinker containing a thiol moiety. In more preferred embodiments, the crosslinker is a heterobifunctional crosslinker. Heterobifunctional crosslinkers react with the surface monolayer via a different mechanism or reaction chemistry than the reaction used to link target molecules to the crosslinker. In still more preferred embodiments, the thiol moiety is a reversibly protected thiol moiety. The preferred protected heterobifunctional crosslinker for creating disulfide attachments is N-succinimydyl S-acetylthioproprionate (SATP).

In the preferred embodiment, SATP is reacted with an amine-terminated surface, for example, a MUAM monolayer. 2.0 mM SATP in 10% N,N-dimethylformamide and 90% 0.1M TEA buffer solution pH 7.0 is placed in contact with the amine-terminated monolayer and allowed to react for 1-2 h. The SATP-terminated surface is deprotected by soaking in a solution of 0.5 hydroxylamine, 0.05 M dithiothreitol (DTT), 0.05 M phosphate buffer, and 0.025 M ethylenediaminetetraacetic acid (EDTA), pH 7.5 for 20 minutes. This results in creation of a sulfhydryl-terminated surface for attachment of reactive target molecules.

In one embodiment, the reactive target molecules contain a maleimide moiety. The sulfhydryl moieties are reacted with a 10 mM solution of the maleimide-containing target molecule in 0.1M triethanolamine hydrochloride (TEA) pH 7.0 for 30 minutes.

In a more preferred embodiment, the sulfhydryl-terminated surface is reacted with 2,2′-dipyridyl disulfide (1 mg/ml) dissolved in a 1:1 mixture of 0.1 M TEA pH 8.0 and N,N′-dimethylformamide (DMF) for two hours, creating a disulfide moiety on the surface. Thiol-containing molecules can then be reacted with the disulfide surface. 1 mM thiol-modified target molecules in 0.1 M TEA buffer pH 8.0 are reacted with the disulfide-terminated surface. The target solution is placed on the surface and covered with a glass slide. The reaction is allowed to proceed in a humid chamber for 6 h. Next, the slide is immersed in a 20 mM sodium phosphate, 100 mM NaCl, 1 mM EDTA solution to remove any nonspecifically adsorbed target molecules. The disulfide bond linking the target molecules to the surface may be cleaved by reaction with 50 mM DTT in 0.1 M TEA, pH 7.0.

In some embodiments, a background resistant to non-specific biomolecule adhesion is created outside of the intended target molecule attachment regions. In a preferred embodiment, a monolayer of 1-octadecanethiol (ODT) is first deposited on a gold-coated surface from a 1.0 mM ethanolic solution for at least 24 h. Next, the bond anchoring the alkanethiol to the metal substrate is selectively cleaved to yield a patterned surface of exposed metal. In some preferred embodiments, UV photopatterning is utilized to create the patterned surface. However, any suitable method of generating a patterned surface may be utilized. For example, in some embodiments, microcontact printing methods can also be used to yield a patterned surface. Using UV patterning, the surface is exposed through a quartz mask to UV radiation, which photo-oxidizes the gold-sulfur bond that anchors the alkanethiol monolayers to the surface. The surface is then rinsed, removing the photo-oxidized alkanethiol and leaving an array of bare metal pads surrounded by a hydrophobic ODT background. Using photopatterning, features with dimensions as small as 50 nm have been achieved; using microcontact printing methods, arrays with features as small as about 100 nm are achievable. The reaction steps described above may then be utilized to fill in the exposed metal pads and create a DPDS disulfide-terminated surface.

The proteins to be arrayed are, in certain embodiments, provided in substantially pure form in solutions. These solutions can be provided in nanoliter-scale volumes ranging from about 1 nL to about 1000 nL, in certain embodiments about 1 nL, and the protein solutions are delivered to the slide, yielding spots approximately 150-200 μm in diameter. In certain embodiments of special interest, the proteins to be arrayed are provided in a buffered aqueous solution containing a humectant (e.g., glycerol, polyethylene glycol) to prevent evaporation of the nanodroplets. The proteins should remain hydrated throughout the preparation, storage, and assaying of the array to prevent denaturation of the protein. The proteins are then contacted with the solid support facilitating attachment through the chemically active sites on the support. The sites on the array which do not contain arrayed protein may be blocked using another protein or molecule. In certain embodiments, BSA, caseine, nonfat milk, glycine, or ethanolamine is used to block the microarray.

In one exemplary embodiment, the blocking of aldehyde slides with BSA serves not only to quench the unreacted groups on the support, but also to form a molecular layer of BSA that reduces nonspecific binding of other proteins to the surface in subsequent steps. In certain preferred embodiments, the immobilized and arrayed proteins are functional and retain a substantial fraction of their original activity. The preparation of solid supports uniformly coated with a protein may be prepared using any techniques known in the art of coating.

3. Microfluidics

In some embodiments, arrays are fabricated by patterning the solid support with microfluidic channels. In some embodiments, microfluidics are generated using the polydimethylsiloxane (PDMS) polymer-based methods described by Lee et al. (Analytical Chemistry, 73:5525 [2001]). This technique can be used for both fabricating 1-D DNA microarrays using parallel microfluidic channels on chemically modified gold and silicon surfaces, and in a microliter detection volume methodology that uses 2-D DNA microarrays formed by employing the 1-D DNA microarrays in conjunction with a second set of parallel microfluidic channels for solution delivery.

For example, in some embodiments, microliter detection volume methodology that uses 2-D DNA hybridization on microarrays formed by employing 1-D DNA line arrays in conjunction with a second set of parallel microfluidic channels for solution delivery is utilized. In some embodiments, PDMS microchannels are fabricated by replication from 3-D silicon wafer masters that were created photolithographically from 2-D chrome mask patterns (See e.g., Duffy et al., Anal. Chem., 70:4974 [1998] and Effenhauser et al., Anal. Chem., 69:3451 [1997]).

A gold thin film surface deposited on the solid support is reacted with MUAM in order to form a self-assembled monolayer on the gold surface as described above. A PDMS polymer film containing parallel microchannels is then attached to the MUAM modified gold surface. In some embodiments, a surface pattern is created by flowing the heterobifunctional linker SSMCC through the PDMS microchannels over the gold surface. The SSMCC reacts with the MUAM to create a maleimide-terminated alkanethiol monolayer. Biological macromolecules (e.g., 5′-thiol-modified DNA or RNA probes) are then each flowed into a separate PDMS microchannel and react with the maleimide-terminated gold surface to form an array of probes on the surface of the gold. In some embodiments, the microchannels are cleaned with water, the PDMS is removed from the surface and the gold slide is soaked in a PEG-NHS solution in order to modify the MUAM background (see above description of blocking with PEG-NHS). The PEG-coated background helps to eliminate nonspecific adsorption of DNA, RNA or protein during hybridization experiments.

The present invention is not limited to a particular method of fabricating channels in the solid surfaces of the present invention. For example, in other embodiments, the present invention utilizes microchannels etched into the prism (See e.g., U.S. Pat. No. 6,176,962, herein incorporated by reference). In still further embodiments, microfluidic channels are fabricated using wet chemical etching (Wang et al., Anal. Chem., 72:2514 [2000]) or soft lithography (Deng et al., Anal. Chem. 72:3176 [2000]).

4. Array Processing

In some embodiments, following patterning or generation of arrays, a silicone gasket (Grace Biolabs, Bend, Or) is sandwiched in-between the solid surface and a microscope cover slide to form a small reaction chamber. In other embodiments, a HYBRIWELL seal (Grace Biolabs) is used to create a low-volume reaction chamber.

II. Cell Adhesion Assays on a Solid Support The present invention provides compositions and methods for the detection of whole cell solution, lysed cell solution, and subcellular compartment interaction with target molecules. The below description provides several exemplary non-limiting methods.

A. Cells

The present invention is not limited to the analysis of a particular cell type. Specific cells from any organism may be utilized. In some embodiments, the contemplated cell type is keratinizing epithelial cells (e.g., basal cell of epidermis, hair shaft cell, basal cell of nail bed). In other embodiments, the cell type may be cells of the wet stratified barrier epithelia (e.g., surface epithelial cells, cells of the urinary epithelium); epithelial cells specialized for exocrine secretion (e.g., salivary gland cells, mammary gland cells, apocrine sweat gland cells, mucous cells of the stomach lining); cells specialized for secretion of hormones (e.g., secreting cells of the pituitary gland, secreting cells of the gut and respiratory tract, secreting cells of the thyroid gland, secreting cells of the adrenal gland, secreting cells of the gonads); epithelial absorptive cells in gut, exocrine glands, and urogential tract (e.g., brush border cells of the intestine, striated duct cells of the exocrine glands, nonciliated cells of the ductulus efferens); cells specialized for metabolism and storage (e.g., hepatocytes, fat cells); epithelial cells serving primarily a barrier function, lining the lung, gut, exocrine glands, and urogenital tract (e.g., type 1 pneumocytes, pancreatic duct cells, parietal cells of kidney glomerulus); epithelial cells lining closed internal body cavaties (e.g., vascular endothelial cells of blood vessels and lymphatics, synovial cells, cells lining endolymphatic space of ear, corneal “endothelial” cells); ciliated cells with propulsive function (e.g., respiratory tract cells, oviduct cells, ependymal cell lining of brain cavaties); cells specialized for secretion of extracellular matrix (e.g., ameloblast cells, fibroblasts, pericytes of blood capillaries, chondrocytes, osteoblasts); contractile cells (e.g., skeletal muscle cells, heart muscle cells, smooth muscle cells, myoepithelial cells); cells of blood and immune system (e.g., red blood cells, macrophages, neutrophils, T lymphocytes, B lymphocytes); sensory transducers (e.g., photoreceptors, inner hair cell of organ of Corti, type II taste bud cells); autonomic neurons (e.g., cholinergic cells, adrenergic cells, peptidergic cells); supporting cells of sense organs and of peripheral neurons (e.g., inner pillar cells, Hensen cells, Schwann cells, enteric glial cells); neurons and glial cells of central nervous system (e.g., neuronal cells in general, astrocytes, oligodendrocytes); lens cells (e.g., anterior lens epithelial cell, lens fiber cell); pigment cells (e.g., melanocytes, retinal pigmented epithelial cells); germ cells (e.g., oogonium cells, oocytes, spermatocytes, spermatogonium); nurse cells (e.g., ovarian follicle cells, Sertoli cells, thymus epithelial cell). In yet other embodiments, unknown cell cultures are screened with known target molecules for cell identity.

In some preferred embodiments of the present invention, stem cells are analyzed. The present invention is not limited to the analysis of a particular stem cell type. Specific stem cells from any organism may be utilized. In some preferred embodiments, the stem cell type may be adult stem cells (e.g., somatic stem cells). In other preferred embodiments, the stem cell type may be embryonic stem cells (e.g., totipotent stem cells).

Stem cells (See e.g., U.S. Pat. Nos. 6,326,198, 6,245,566, and 6,200,806, WO 00/27995, WO 00/73421, WO 01/68815, WO 01/96532, WO 00/12682, and WO 01/53465, each of which is herein incorporated by reference) are undifferentiated cells that can give rise to a succession of mature functional cells.

Stem cells have the capacity, upon division, for both self-renewal and differentiation into progenitors. Thus, dividing stem cells generate both additional primitive stem cells and somewhat more differentiated progenitor cells. In addition to the generation of blood cells, stem cells also may give rise to osteoblasts and osteoclasts, and cells of other tissues.

A variety of strategies have been proposed for providing stem cells. These strategies may be divided into two different groups—stem cells derived from embryonic sources and stem cells derived from adult sources. For example, a hematopoietic stem cell may give rise to any of the different types of terminally differentiated blood cells. Embryonic stem (ES) cells are derived from the embryo and are totipotent, thus possessing the capability of developing into any organ or tissue type or, at least potentially, into a complete embryo. For example, U.S. Pat. No. 5,843,780 to Thompson describes the production of stem cell lines from human embryos. PCT publications WO 00/52145 and WO 01/00650 describe the use of cells from adult humans in a nuclear transfer procedure to produce stem cell lines.

Examples of adult stem cells include hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, cells from all three germ layers (adult neural stem cells).

In order to retain the stem cell phenotype during in vitro culture, stem cells are frequently cultured on a feeder layer of fibroblasts (such as murine 3T3 or STO cells, See e.g., Martin and Evans, Proc. Natl. Acad. Sci USA 72:1441-1445 [1975]).

B. Target molecules

In some embodiments, target molecules (e.g., enzymes, oligonucleotides (e.g., containing a particular DNA binding domain recognition sequence), viruses, polypeptides, antibodies, naturally occurring drugs, synthetic drugs, pollutants, allergens, affector molecules, growth factors, synthetic molecules, chemokines, cytokines, lymphokines, nucleic acids) are attached to surfaces configured for label-free (e.g., SPR) detection. Target molecules are contemplated to comprise proteins, enzymes, or other arrangements of proteins. In some preferred embodiments, arrays of molecules are attached to the solid surfaces. In some embodiments, multiple copies of the same molecule targets are attached to different places on the array. In other embodiments, different target molecules are attached to each place on the array. In some embodiments, the target molecule is known. In still further embodiments, randomized target molecules are attached to arrayed surfaces.

The present invention is not limited to any particular type of molecule. Specific molecules from any organism or synthetic (e.g., drugs, mimetics) molecules may be used. In preferred embodiments, targets molecules are suspected or known to perform a role in cell development (e.g., differentiation, morphogenesis, maintenance). As such, some embodiments utilize target molecules specific for extracellular matrix proteins (e.g., porin, glycoporin, selectins, lipoprotein, spectrin, ankyrin). In other embodiments the target molecules are specific for membrane transport (e.g., NaK ATPase proteins, Ca Pump proteins, Ion Channel proteins). In other embodiments the target molecules are specific for intracellular compartmentalizing and protein sorting (e.g., various organelle import/export peptide signals; nuclear pore complex proteins, ribosome receptor proteins, SRP receptor proteins). In other embodiments the target molecules are specific for secretory and endocytic pathways (e.g., hydrolase proteins, mannose 6-phosphate receptor, clathrin, LDL receptor protein, adaptins, coatomer, ARF, SNAREs, Rab proteins). In other embodiments the target molecules are specific for cell signaling (e.g., eicosanoids, G-proteins, G-protein receptors, adenylyl cyclase, thyroid stimulating hormone, adrenocorticotropic hormone, luteinizing hormone, adrenaline, glucagons, protein kinase C, calmodulin, CaM-kinase II, cyclic AMP, receptor guanylyl cyclases, tyrosine specific protein kinases, EGF receptor, insulin receptor, NGF receptor, PDGF receptor, FGF receptor, VEGF receptor, Ras proteins, MAP kinases, Src kinases, Janus kinases). In other embodiments the target molecules are specific for the cytoskeleton growth and maintenance (e.g., actin filaments, microtubles, intermediate filaments, kinesins, dyneins, keratins, vementin, neurofilament proteins, nuclear lamins, tubulin, thymosin, profiling, spectrin, ankyrin, fimbrin, alpha-actinin, gelsolin, myosin, villin, tropomyosins, filamin). In other embodiments the target molecules are specific for cell division (e.g., cyclin dependent protein kinases, mitotic cyclins, GI cyclins, maturation promoting factor, Cdc2, cyclin B, cyclin E, platelet derived growth factor, EGF, IGF-I, TGF-beta, FGF, IL-2, NGF, IL-3, kinetochore microtubules, astral microtubules). In other embodiments the target molecules are specific for cell junction, cell adhesion and the extracellular matrix (e.g., cadherins, catenins, vinculin, alpha-actinin, plakoglobin, integrin, connexon, connexin, glycosaminoglycans, proteoglycans, fibronectin, collagen, laminin).

In preferred embodiments the target molecules are specific for various mechanisms of cell and stem cell development (e.g., cleavage, gastrulation, anterior/posterior axis formation, dorsal/ventral formation, central nervous system formation, neural crest specificity, mesoderm formation, lateral plate formation, limb formation, sex determination, metamorphasis processes). In some preferred embodiments, the target molecules are specific for cleavage mechanisms (e.g., morula molecules, trophoblast molecules, chorion molecules, placenta molecues, inner cell mass molecules, strypsin, collagen, laminin, fibronectin, hyaluronic acid, heparin sulfate receptors, integrins, collegnase, stromelysin, plasminogen activator). In other preferred embodiments the target molecules are specific for gastrulation processes (e.g., nodal molecules, primitive endoderm molecules, extraembryonic endoderm molecules, embryonic epiblast molecules, amnionic cavity molecules, amnionic fluid molecules). In other preferred embodiments, the target molecules are specific for anterior/posterior axis formation (e.g., anterior visceral endoderm molecules, chordin, noggin, Hox proteins). In other preferred embodiments, the target molecules are specific for left-right axis development (e.g., inv, nodal, lefty-2, pitx2, snail, neural tube molecules). In other preferred embodiments, the target molecules are specific for central nervous system development (e.g., neural tube molecules, neural plate molecules, spinal cord molecules, optic molecules, cutaneous structure molecules). In other preferred embodiments, the target molecules are specific for neural crest and axonal development (e.g., cranial neural crest molecules, trunk neural crest molecules, vagal neural crest molecules). In other preferred embodiments the target molecules are specific for paraxial and intermediate mesoderm development (e.g., somite molecules, muscle formation molecules, bone development molecules, kidney development molecules). In other preferred embodiments the target molecules are specific for lateral plate mesoderm and endoderm development (e.g., cardiogenic mesoderm molecules, vasculogenesis molecules, angiogenesis molecules, gut tissue development molecules, respiratory development molecules). In other preferred embodiments the target molecules are specific for limb development (e.g., Tbx5, Tbx4, apical ectodermal ridge molecules, FGF4, FGF8, shh, Hox proteins, sonic hedgehog, ZPA, Wnt). In other preferred embodiments the target molecules are specific for sex determination (e.g., SF1, WT1, LHX9, DAX1, WNT4a, SRY, SOX9). In other preferred embodiments the target molecules are specific for cellular metamorphasis (e.g., Wg, Dpp, Distal-less, Dpp, hedgehog, engrailed, Wingless).

In other embodiments, a cell's molecules are known but the particular target molecules are unknown. In still further embodiments, mutants or variants of known target molecules are utilized.

In some embodiments, small target molecules are attached to solid surfaces (e.g., as arrays of small target molecules). In some embodiments, multiple copies of the same target molecule are attached as arrays on solid surfaces. In other embodiments, different target molecules are attached to each location of an array on a solid surface.

C. Cell Adhesion Assays

In some embodiments, whole cell solutions are exposed to an array of target molecules on the surface of a solid support. In other embodiments, lysed cell solutions are exposed to an array of target molecules on the surface of a solid support. In yet other embodiments, subcellular compartments of a cell are exposed to an array of target molecules on the surface of a solid support. In some embodiments, the same target molecule is applied at multiple addresses in the array. In some embodiments, each target molecule is tested by directly spotting experimental solutions on the probe. Alternatively, in other embodiments, each target molecule is tested by using microfluidics to deliver the sample to individual probe spots.

In some embodiments, microfluidics are utilized to deliver the whole cell solution, lysed cell solution, or subcellular compartment to the individual spots on the array. The binding affinity is compared in the presence of various test compounds to the binding in the absence. Binding of target molecules is detected using any suitable method (e.g., those described below).

One preferred embodiment for the cell adhesion assay is described in Bioconjugate Chem. 2001, 12, 346-353 (herein incorporated by reference). The presence of cells adhering to a surface is detected by calcein AM-labeling of the cells, and with Giemsa staining of cells. Detection is performed with a fluorescence microscope. In some embodiments, detection could be performed with an array reader, or a surface plasmon resonance based device. Non specific binding of the cells to the background will be reduced by modifying the surface with the NHS ester of methoxypoly (ethylene glycol) propionic acid (PEG-NHS) (preferably of molecular weight over 5000) attached to an amine modified gold, silicon, diamond, or other surface.

D. Detection Methods

The binding of a cellular item to a molecule target may be detected using any suitable method, including but not limited to, label free detection, detection of a label, or a combination method. In some embodiments, binding is quantitated.

1. Label Free Target Molecule Detection

In some preferred embodiments, detection is label free. For example, in some embodiments, SPR (See e.g., above description) is utilized. In other embodiments, the label free electrical detection method described in WO 01/61053A2 (herein incorporated by reference) is utilized.

In still further embodiments, oligonucleotide-conjugated nanoparticles are utilized for detection of binding (See e.g., Nanosphere, Northbrook, Ill., U.S. Pat. No. 6,361,944, herein incorporated by reference). The assay involves a detectable change (e.g., a color change, the formation of aggregates of the nanoparticles, or the precipitation of the aggregated nanoparticles) that occurs upon hybridization of the oligonucleotides on the nanoparticles to the target molecule. The color changes can be observed with the naked eye or spectroscopically. The formation of aggregates of the nanoparticles can be observed by electron microscopy or by nephelometry. The precipitation of the aggregated nanoparticles can be observed with the naked eye or microscopically.

In some embodiments, target molecule arrays are labeled with nanoparticles and detected using the described methods. In other embodiments, following binding of a cellular item to a bound target, cellular items not bound to a target are detected by oligonucleotide-nanoparticle conjugates (e.g., the absence of a signal is indicative of a positive binding event). In still further embodiments where a target molecule is bound to the solid support, bound cellular items are detected using the conjugates (e.g., by an oligonucleotide-nanoparticle that is complementary to the target molecule).

2. Additional Detection Methods

In some embodiments, detection via a label is utilized. In some embodiments, detection via a label is combined with label free (e.g., SPR) detection methods. In other embodiments, detection with a label is utilized independent of label-free detection.

Detection thresholds are often a limiting factor in SPR detection methods. SPR detection is primarily a function of changes in mass adherent to the SPR surface. Methods that greatly increase or decrease the bound mass affect sensitivity. Thus, in some embodiments, additional detection methods are utilized to alter the mass of the complex being detected.

For example, in some embodiments, whole cell solutions, cellular fragments, or subcellular components are mixed with target molecules in a vessel. The target molecules are allowed to bind with the cell. Unbound target molecules are separated from bound target molecules. Bound complexes are then detected by hybridization to a second target molecule arrayed on an SPR-capable surface. In some embodiments, the first target molecule has a ‘sticky end’ that protrudes from the target molecule binding site. This sticky end hybridizes to the complementary second target molecule on the array surface. The bound mass is therefore increased by the mass of the primary DNA probe. In some embodiments, the mass is further increased by conjugation of the target nucleic acid to a label or nanoparticles (e.g., gold).

In other embodiments, antibodies are utilized for enhancing the SPR signal generated by cellular item—target molecule complexes. The cellular item directly binds to the arrayed target molecule. In some embodiments, the SPR signal is then enhanced by binding of an antibody to the target molecule. In some embodiments, the antibody is labeled (e.g., with fluorescent labels (e.g., fluorescein), enzymatic detection labels (e.g., horse radish peroxidase), and metal labels (e.g., gold)). This method has the further advantage of immunologically confirming the identity of the protein binding to the target molecule.

3. Quantitation

In some preferred embodiments, binding assays are quantitative. For example, the detection methods of the present invention allow for investigation of the kinetics of binding. Kinetic measurements are obtained by taking multiple time points and analyzing the rate of increase in signal.

III. Applications of Binding Assays

The binding assay of the present invention finds use in a variety of applications, including, but not limited to, the identification of proteins involved in various cellular processes, the screening of growth conditions, and screening of test compounds.

A. Identification of Proteins Involved in Various Cellular Processes

In some embodiments, the present invention provides methods for identifying proteins involved in various cellular processes. In this embodiment, arrays are addressed such that sites giving a positive binding signal can be identified. As such, it is contemplated that in the construction of arrays, target molecules will be distributed in a known manner.

In other embodiments, label-free (e.g., SPR) imaging is used to measure binding to thousands of arrayed target molecules simultaneously. Thus, the present invention provides methods of identifying binding sites for target molecules without any prior knowledge of the recognition site.

B. Screening for Growth Conditions

In other embodiments, the methods of the present invention are utilized to measure binding of target molecules from cell extracts. Such information is useful to researchers studying regulation of particular cells. In some preferred embodiments, whole cell solutions, lysed cell solutions, subcellular compartments, or mixes therewith, are exposed to two different conditions (e.g., media, temperature, antibiotics, etc.) and differences in binding resulting from this treatment are observed. In other preferred embodiments, whole cell solutions, lysed cell solutions, subcellular compartments, or mixes therewith, at different stages of cellular development, are exposed to arrays of target molecules. The methods of the present invention provide the added advantage that whole suites of target molecules can be screened for binding simultaneously. This knowledge is particularly suited for the understanding of regulatory networks in cells. In some embodiments, sensitivity and cell extract volume requirements are enhanced by using microfluidics or targeted spotting of array features to apply the cell extract to particular surface addresses.

C. Screening Test Compounds

In still further embodiments, the methods of the present invention are used to measure the effect of small molecule test compounds including, but not limited to, drugs (e.g., cancer drugs, suspected carcinogens, antibiotics, growth conditions, and cell type) on the target molecule binding affinity. For example, in some embodiments, a whole cell product is prepared and divided into two aliquots. The test compound is added to one aliquot, but not the other, and purified cell product is mixed with both. The change in state of the target molecule (bound vs. unbound or the affinity of binding) is then measured by observing cell product binding to the arrayed target molecules. The effect of the molecule on activation or inactivation of multiple target molecules is assayed simultaneously using such arrays.

In other embodiments, the effect of the molecule on target molecule binding is assayed directly without the use of cell extracts. This is useful for target molecules that can be switched on or off using simple in vitro reactions, such as phosphorylation. This is also useful when compounds that alter target molecule binding are sought. In a particular embodiment, a compound that disrupts binding of growth factors to cancer cells is identified in this manner.

In still further embodiments, arrays of target molecules are exposed to test compounds. In some embodiments, test compounds are removed from the array prior to testing. In other embodiments, test compounds are left on the array during testing. The array is then contacted with whole cell solutions, lysed cell solutions, subcellular compartments, or mixes therewith, and the binding measured.

D. Binding Assay Kits

In some embodiments, the present invention provides kits for performing the process described herein. In preferred embodiments, the kits allow end-users to attach their own target molecule content, and then assay that content against the end-users cell product of interest. Such a kit would include a prepared surface for arraying the target molecules, including any additional attachment chemistry (e.g., a gold-coated slide, preferably with a MUAM monolayer, a crosslinker, blocking agent [a PEG compound or BSA], preferably a general buffer set, and instructions for preparing the surface, stains (Giesma, etc.), dyes or probes, etc., attaching the target molecule content, performing the interaction assay, and readout of the results. Alternatively, the kits of the present invention may comprise a target molecule array, plus buffers, etc.

All publications and patents mentioned in the above specification are herein incorporated by reference. Various modifications and variations of the described devices, compositions, methods, systems, and kits of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in art are intended to be within the scope of the following claims.

We claim:

1. An apparatus comprising an arrayed solid surface; said solid surface comprising a plurality of addressable target molecules; said solid surface in contact with a cellular product.

2. The apparatus of claim 1; wherein said target molecules are selected from the group consisting of natural or synthetic oligonucleotides, viruses, polypeptides, antibodies, naturally occurring drugs, synthetic drugs, pollutants, allergens, affector molecules, growth factors, chemokines, nucleic acids, cytokines, and lymphokines.

3. The apparatus of claim 1; wherein said plurality of target molecules are selected from group consisting of cell initiation molecules, cell differentiation molecules, cell morphogenesis molecules, and cell maintenance molecules.

4. The apparatus of claim 1; wherein said cellular product is selected from the group consisting of whole cell solutions, lysed cell solutions, and subcellular compartment solutions.

5. The apparatus of claim 1; wherein said cellular product comprises a cellular mixture;
said cellular mixture comprising combinations of whole cell solutions, lysed cell solutions, or subcellular compartment solutions.

6. The apparatus of claim 1; wherein said cellular product is selected from the group consisting of whole stem cell solutions and lysed stem cell solutions.

7. The apparatus of claim 1; wherein said solid surface is configured for label free detection.

8. The apparatus of claim 1; wherein said solid surface is an SPR surface.

9. The apparatus of claim 1; wherein said solid surface further comprises a plurality of microfluidics channels.

10. The apparatus of claim 1; wherein said plurality of target molecules comprises at least 2 unique target molecules.

11. The apparatus of claim 1; wherein said plurality of target molecules comprises at least 50 unique target molecules.

12. The apparatus of claim 1; wherein said plurality of target molecules comprises between 10 and 10000 unique target molecules.

13. A method of identifying molecules that interact with a cell component, comprising:
a) providing
i) an arrayed solid surface, said solid surface comprising an array of addressable target molecules; and
ii) a cellular product; and
b) contacting said cellular product with said arrayed solid surface.

14. A system comprising an arrayed solid surface, said solid surface comprising a plurality of addressable target molecules; a cellular product, said cellular product in contact with said arrayed solid surface.

15. The system of claim 14; further comprising a detection apparatus; said detection apparatus configured to detect the presence or absence of an interaction between said cellular product and said target molecules.

16. The system of claim 14; further comprising an instruction manual.

17. A compound, produced by the process comprising:
a. providing
i. a solid surface, said solid surface comprising an array of addressable putative signal molecules;
ii. a cellular product; and
b. contacting said solid surface with said cellular product.
c. identifying said putative signal molecule.
